(19) United States
(12) Patent Application Publication
(10) Pub. No.: US 2010/0010279 A1
(43) Pub. Date: Jan. 14, 2010

(54) Catalyst Compositions Comprising Metal Phosphate Bound Zeolite and Methods of Using Same to Catalytically Crack Hydrocarbons

(76) Inventor: Ranjit Kumar, Clarksville, MD (US)

Correspondence Address: W.R. GRACE & CO.-CONN., 7500 GRACE DRIVE, COLUMBIA, MD, 21044, US

(21) Appl. No.: 12/502,865

(22) Filed: Jul. 14, 2009

Related U.S. Application Data
(63) Continuation-in-part of application No. 10/817,069, filed on Apr. 2, 2004, now abandoned.

(51) Int. Cl. B01J 29/40; B01J 29/06; B01J 23/10; B01J 29/072; B01J 27/182; C07C 4/06

(52) U.S. Cl. 585/653; 502/64; 502/65; 502/66; 502/68; 502/71; 502/214

(57) ABSTRACT

A catalyst composition comprising metal phosphate binder and zeolite can be used to enhance olefin yields during hydrocarbon cracking processes. The composition typically further comprises aluminum phosphate, and the metal of the metal phosphate is a metal other than aluminum. Depending on the metal chosen, enhanced propylene and isobutylene yields in fluid catalytic cracking processes can be obtained compared to catalysts that do not contain such metal phosphate binders. The catalyst can also comprise non-zeolitic molecular sieves, thereby making the composition suitable for use in areas outside of catalytic cracking, e.g., purification and adsorbent applications.

### RELATED APPLICATION

This application is a continuation-in-part application of U.S. patent application Ser. No. 10/817,069, filed Apr. 2, 2004.

### BACKGROUND

The present invention relates to improved catalysts, and more specifically to catalytic cracking catalysts comprising zeolite and metal phosphate that are particularly selective for the production of C₃ and C₄ olefins.

Catalysts and zeolites that include a phosphorus component are described in the following references.
U.S. Pat. No. 3,354,096 describes zeolite-containing adsorbent and catalyst compositions that contain a phosphate binding agent to improve physical strength.

U.S. Pat. No. 3,649,523 describes hydrocracking catalysts that comprise a zeolite and an aluminum phosphate gel matrix.

U.S. Pat. Nos. 4,454,241, 4,465,780, 4,498,975 and 4,504,382 describe zeolite catalysts that are prepared from clay which are further modified by the addition of a phosphate compound to enhance catalytic activity.

U.S. Pat. Nos. 4,567,152, 4,584,091, 4,629,717 and 4,692,236 describe zeolite-containing catalytic cracking catalysts that include phosphorus-containing alumina.

U.S. Pat. Nos. 4,605,637, 4,578,371, 4,724,066 and 4,839,319 describe phosphorus and aluminum phosphate modified zeolites such as ZSM-5, Beta and ultrastable Y that are used in the preparation of catalytic compositions, including catalytic cracking catalysts.

U.S. Pat. No. 4,765,884 and U.S. Pat. No. 4,873,211 describe the preparation of cracking catalysts which consist of a zeolite and a precipitated alumina phosphate gel matrix.

U.S. Pat. No. 5,194,412 describes preparing a cracking catalyst that contains zeolite and an aluminum phosphate binder.

### SUMMARY OF THE INVENTION

It is an object of the present invention to provide improved catalytic compositions, especially fluidized cracking catalysts, that comprise a zeolite, aluminum phosphate and metal phosphate that is present in an amount sufficient for it to at least function as a binder for the zeolite and the metal is other than aluminum.

It is also an object of the present invention to provide improved catalytic compositions that comprise non-zeolitic sieves and metal phosphate that is present in an amount sufficient for it to at least function as a binder for the sieve and the metal is other than aluminum.

It is a further object to provide a method for preparing zeolite/metal phosphate binder-containing cracking catalysts that are selective for the production of light olefins, e.g., C₃ and C₄ olefins, and further, that selectivity is enhanced compared to the activity of catalysts that do not contain such binders.

It is yet a further object to provide a means to manipulate and more easily influence olefin yields from processes of catalytic cracking hydrocarbons. For example, aluminum phosphate binders described in U.S. Pat. No. 5,194,412 and catalysts made from those binders have been shown to be useful in enhancing olefin yields in such processes. The new metal phosphate binders described herein offer additional choices to enhance olefin yields, and catalysts comprising preferred embodiments of the metal phosphate binder of this invention, e.g., iron phosphate, unexpectedly enhance yields with respect to certain olefins.

It is still a further object to provide an FCC process that is capable of producing higher ratios of propylene to butylenes.

It is still a further object to provide an FCC process that is capable of producing lower ratios of propylene to butylenes.

### BRIEF DESCRIPTION OF THE DRAWING

FIG. 1 is a schematic flow diagram that illustrates a suitable process for preparing the catalysts of the present invention.

FIG. 2 is the ³¹P NMR spectrum of the sample (Fe) from Example 1 with peaks at −6, −15, −32, 43, and ˜49 parts per million (ppm), with the −32 peak attributed to an AlPO₄ site.

FIG. 3 is the ³¹P NMR spectrum of the sample (Ca) from Example 2 with peaks at 0, −11, −14, −32, and −43 ppm, with the −32 peak attributed to an AlPO₄ site.

FIG. 4 is the ³¹P NMR spectrum of the sample (Ca) from Example 3 with peaks at 0, −11, −14, −32, and −43 ppm, with −32 peak attributed to an AlPO₄ site.

FIG. 5 is the ³¹P NMR spectrum of the sample (Ca) from Example 4 with peaks at 0, −11, −14, −32, and −43 ppm, with the −32 peak attributed to an AlPO₄ site.

FIG. 6 is the ³¹P NMR spectrum of the sample (Al) from Example 5 with a peak at −32 ppm attributed to an AlPO₄ site.

FIG. 7 is the ³¹P NMR spectrum of the sample (Sr) from Example 6 with peaks at 1, −9, −32, and −43 ppm, with the −32 peak attributed to an AlPO₄ site.

FIG. 8 is the ³¹P NMR spectrum of the sample (La) from Example 7 with peaks at 0, −6, −32, and −43 ppm, with the −32 peak attributed to an AlPO₄ site.

FIG. 9 is the ³¹P NMR spectrum of the sample (Mg) from Example 8 with peaks at −2, −11, −14, −32, and 43 ppm, with the −32 peak attributed to an AlPO₄ site.

### DETAILED DESCRIPTION OF THE INVENTION

The catalyst composition of this invention comprises zeolite and a metal phosphate that is present in an amount sufficient to at least function as a binder for the zeolite. It has been found that these compositions are highly active catalysts suitable for enhancing yields of light olefins when cracking hydrocarbon feed streams.

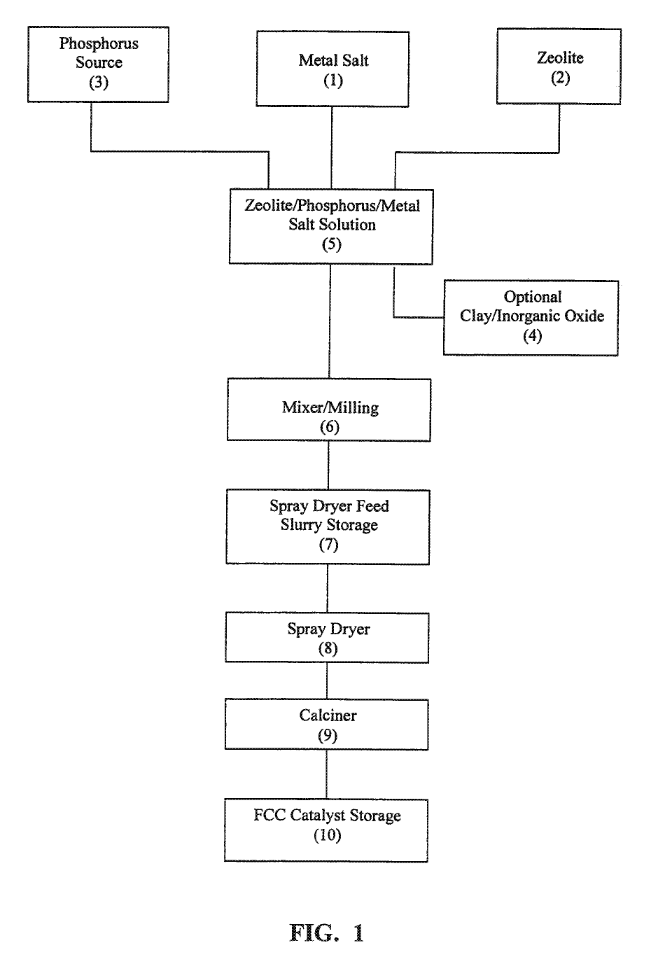

As illustrated in FIG. 1, the catalysts of this invention may be prepared by mixing in water a metal salt (1), which is other than an aluminum salt, and one or more zeolite or sieve (2), and then adding a source of phosphorus (3), e.g., phosphoric acid, and optionally a finely divided particulate inorganic oxide component (4), including, but not limited to, clay and alumina. The resulting slurry (5) can then be processed to obtain bound catalytic composites having desired properties, shape and size. FIG. 1 schematically illustrates processing the resulting slurry in a mixer (6) and spray drier (8) to form the desired bound catalyst composition.

In one embodiment for preparing the catalysts of the present invention, zeolite (2) is added as a powder to an aqueous metal salt solution (1) that is other than an aluminum salt to form a slurry, which said slurry is combined with phosphoric acid solution that serves as the phosphorus source (3). It is also preferable to add clay (4) to the slurry. The resulting slurry is then subjected to high shear mixing and milling conditions at (6) to obtain a spray drier feed slurry that is either stored at (7) and/or spray dried at (8). It is also suitable to add metal salt powder and zeolite powder to a phosphoric acid solution, and then adding additional water to form the zeolite/phosphorus/metal salt solution and slurry (5) prior to adding clay and mixing at (6).

The conditions of adding the aforementioned components and processing the same are selected to form the desired metal phosphate binder in form suitable for use as a catalyst. Such conditions are well known. For example, the pH of the resulting mixture of zeolite, metal salt, phosphorus, and optional clay, other inorganic oxides, and water can be made to have a pH of below 7 preferably below 5 and more preferably below 3. In certain instances, pH's higher than 7 could result in metal phosphate precipitating out of the slurry thereby preventing a binder from being formed when spray dried.

When spray drying the slurry from (5) to form the catalyst, it is common to spray dry the slurry at gas inlet/outlet temperatures of 300° to 400° C. and 100° to 200° C., respectively. The slurry is typically spray dried to have a mean particle size range of 20 to 150 microns and is typically held in a storage container, e.g., such as (10) in FIG. 1, prior to use.

While spray drying is generally used to prepare FCC catalysts, other forming/drying techniques such a pelletizing and extruding may be used to prepare compositions that are useful in other catalytic processes such as hydrocracking, hydrotreating, isomerization, dewaxing, etc. Such catalyst forms can be used in fixed bed and/or moving bed applications. Techniques suitable for extruding and pelletizing these compositions are well known to those skilled in the art. For example, the feed composition into an extruder or pelletizer generally is the same as that for a spray drier, except that the solids content of a spray drier feed is generally higher than the feed paste for an extruder.

Typically, the catalyst of this invention has a total matrix surface of less than 100 m²/g, or more typically less than 70 m²/g, as measured by BET techniques. When an additional porous inorganic oxide matrix component, such as silica, alumina, magnesia or silica-alumina sols or gels, is added to the catalyst, the matrix component of the invention may have a surface area of up to 300 m²/g.

The catalyst of this invention also is generally made to possess a Davison Attrition Index (DI) of 0 to 30, and preferably 0 to 20, and more preferably from 0 to 15 as determined by the Davison Attrition Index Test described as follows.

After being calcined in a muffle furnace for two hours at 538° C., a 7.0 gram sample of catalyst is screened to remove particles in the 0 to 20 micron size range. The particles above 20 microns are then subjected to a 1 hour test in a standard Roller Particle Size Analyzer using a hardened steel jet cup having a precision bored orifice. An air flow of 21 liters a minute is used. The Davison Index is calculated as follows:

$$Davison Index = \frac{ \begin{matrix}Wt . \% 0 \text{-} 20 micron material formed \\ during test\end{matrix} }{Wt . Original 20 + micron fraction}$$

In general, the components selected to use in the above processes should be those that do not invariably prevent formation of the aforementioned metal phosphate binder. The metal selected for the metal salt should be one that reacts with a phosphorus source to form a compound suitable for functioning or otherwise serving as a binder for zeolite. The metal salt, and of course the phosphorus source, should be added in amounts sufficient to prepare a metal phosphate binder for the zeolite. Generally, the amount of phosphorus should be sufficient to convert all of the metal in the salt to phosphate and aluminum in the zeolite to AlPO₄. To insure sufficient conversion, it is usually desirable to include 0.5 to 1.5% excess phosphoric acid when phosphoric acid is used as the phosphorus source. The amount of phosphorus source used to make the invention also depends on whether aluminum-containing materials other than zeolite and clay are present in the composition. Larger amounts of phosphorus are typically added when such aluminum-containing materials are present.

By “binder”, it is meant a material that provides the function of binding together or adhering the various components of the catalyst composition, especially the zeolite, in a manner such that the resulting composition does not readily disintegrate or break up during a catalytic cracking process. The catalyst of this invention is especially suitable for use as a FCC catalyst, and therefore, it is desirable for the composition of this invention to have attrition properties such that the composition does not readily disintegrate under conventional FCC conditions. For the purposes of this invention, it is usually necessary for the metal phosphate to comprise at least 3% by weight of the catalyst composition, as measured by the amount of oxide of the metal in the metal phosphate using ICP. For the purposes of this invention percentages of metal phosphate reported herein are based on the weight % of the metal's corresponding oxide as measured using ICP techniques. Typically, the composition comprises the metal phosphate in an amount ranging from 4 to 50% by weight of the catalyst composition, as determined by the amount of the metal's corresponding oxide.

The metal salt used to make the invention may be metal nitrate, chloride, or other suitable soluble metal salts. The metal salt could also be a mixture of two or more metal salts where the two or more metals are capable of forming phosphates. In such embodiments, it is believed an interpenetrating network of two or more phosphates are formed, with both phosphates serving as binders. The metal salt is combined with a source of phosphorus and zeolite in amounts to obtain a M (is a cation) to PO₄ ratio of 0.5 to 2.0 and preferably 1 to 1.5, a pH of below 7 and preferably below 5, more preferably below 3, and a solid concentration of 4 to 25 wt. % as metal phosphate. Generally, the metal is selected from the group consisting of Group IIA metals, lanthanide series metals, including scandium, yttrium, lanthanum, and transition metals. Preferred metals include iron (ferric or ferrous being suitable), lanthanum and calcium. In other embodiments Group VIII metals are suitable. In general, the metal salt is usually in the form of a metal salt solution when combining it with the zeolite. However, as mentioned above, it is also suitable to add the metal salt as a powder to the phosphoric acid solution and then later adding water to adjust the concentration of the metal salt to the desired levels.

The phosphorus source should be in a form that will ultimately react with the aforementioned metal to form a metal phosphate binder. For example, the phosphorus source in typical embodiments should be one that remains soluble prior to being spray dried. Otherwise, if the phosphorus source or its resulting phosphate precipitates out of solution prior to spray drying, it will not result in a binder being formed during spray drying. In typical embodiments, the phosphorus source will be phosphoric acid. Another suitable phosphorus source is (NH₄)H₂PO₄.

The zeolite may be any acid resistant zeolite, or a mixture of two or more zeolites, having a silica to alumina molar ratio in excess of about 8 and preferably from about 12 to infinity. Particularly preferred zeolites include zeolite Beta, ZSM zeolites such as ZSM-5, ZSM-11, ZSM-12, ZSM-20, ZSM-23, ZSM-35, ZSM-38, ZSM-50, ultrastable Y zeolite (USY), mordenite, MCM-22, MCM-49, MCM-56, and/or cation, e.g, rare-earth cation, exchanged derivatives thereof. ZSM-5 is a particularly preferred zeolite and is described in U.S. Pat. No. 3,702,886. Zeolite Beta is described in U.S. Pat. No. 3,308,069, and ultrastable Y zeolite is described in U.S. Pat. Nos. 3,293,192 and 3,449,070.

The binder of this invention can also be used to bind non-zeolitic molecular sieves, optionally as mixtures with zeolitic sieves mentioned above. Suitable non-zeolitic sieves include, but are not limited to, SAPO, AlPO, MCM-41, and mixtures thereof.

The zeolite and/or sieve may be slurried first with water prior to adding the metal salt. The zeolite and/or sieve may be added as a powder to phosphoric acid or a metal salt solution.

While clay, such as kaolin clay having a surface area of about 2 to 50 m²/g, is optional, it is preferably included as a component of catalysts designed for FCC processes. The catalyst of this invention may also comprise additional finely divided inorganic oxide components such as other types of clays, silica, alumina, silica-alumina gels and sols. Other suitable optional components include yttria, lanthana, ceria, neodymia, samaria, europia, gadolinia, titania, zirconia, praseodymia and mixtures thereof. When used, the additional materials are used in an amount which does not significantly adversely affect the performance of the compositions to produce olefins under FCC conditions, the hydrocarbon feed conversion or product yield of the catalyst. Typical amounts of additional materials that can be present in the invention range from 0 to about 25% by weight of the total composition.

The catalyst may also comprise binders in addition to the aforementioned metal phosphate. For example, materials can be added to the mixture in mixer (6) of FIG. 1 such that a second binder is formed in addition to the metal phosphate binder. Suitable additional binders include, but are not limited to, colloidal alumina, colloidal silica, colloidal aluminum silicate and aluminum phosphate such as the aluminum phosphate binders described in U.S. Pat. No. 5,194,412. With respect to the preparing a second binder of aluminum phosphate, aluminum phosphate binder precursors are added to mixer (6) and the aluminum phosphate binder forms at about the same time as the metal phosphate binder described herein. The colloidal based binders are generally formed by adding the colloidal dispersions to the mixture in (6).

The metal phosphate formed during the processing stages (6) through (8) of FIG. 1 is set as a binder when the composition is exposed to temperatures of at least 200° C. Therefore the binder of this invention is typically formed by calcining the processed, e.g., spray dried, composition at temperatures of at least 200° C., and preferably at a temperature in the range of 400° to 800° C. Formation of the metal phosphate binder can be confirmed by the presence of a metal-phosphate bond as shown in an NMR analysis run under conditions described later below. In typical embodiments of the invention, the catalyst composition is calcined after spray drying and prior to the catalyst being used, e.g., as illustrated at (9) in FIG. 1. In certain other embodiments, however, the composition may not be calcined prior to being used. In those embodiments the metal phosphate binder is set when it is exposed to the temperatures prevailing during the catalytic process, and any subsequent catalyst regeneration processes. However, caution should normally be taken to avoid exposing an uncalcined composition to water prior to use. Exposure of these embodiments to significant amounts of water prior to use will likely lead to significant disintegration of the composition.

In typical embodiments, the catalyst composition contains relatively small amounts of aluminum phosphate, i.e. regardless of whether a second binder comprising aluminum phosphate is employed. In typical embodiments, the composition contains silica- and alumina-containing zeolites, and it is believed that during the manufacture of the invention, zeolite is dealuminated and the resulting alumina will react with the phosphorus in the phosphorus source to form aluminum phosphate. The amount of aluminum phosphate present therefore depends on how much aluminum is present in the zeolite. For example, compositions of this invention containing low silica to alumina ratio zeolites can have more aluminum phosphate than embodiments containing relatively high silica to alumina ratio zeolites. Alumina can also be present in optional binders and/or additives, e.g., colloidal alumina, and alumina in these materials can also provide source of aluminum to form aluminum phosphate. Unless added as a secondary binder or sieve, the amount of aluminum phosphate generally will be less than the amount of metal phosphate binder present in the catalyst composition. In typical embodiments, the catalyst contains less than 10% by weight aluminum phosphate. Indeed, in certain embodiments where non-zeolitic sieves are used, and there are no binders other than the aforementioned metal phosphate, the amount of aluminum phosphate could be essentially zero.

A typical catalyst composition prepared for use in FCC processes will include the following range of ingredients:

| | |
|---|---|
| Metal Phosphate | 4 to 50 wt. % |
| (Measured As Metal Oxide) | |
| Zeolite and Optional | 2 to 80 wt. % |
| Molecular Sieve: | |
| Optional Inorganic Solid: | 0 to 88 wt. % |

Preferred FCC catalysts under this invention contain from about 5 to 60 wt. % ZSM 5, 0 to 78 wt. % kaolin, and 4 to 40 wt. % metal phosphate.

The catalyst may be used in a conventional FCC unit wherein the catalyst is reacted with a hydrocarbon feedstock at 400° to 700° C. and regenerated at 500° to 850° C. to remove coke. The feedstocks for such processes include, but are not limited to, gas-oil, residual oil and mixtures thereof which may contain up to 10 wt. % Conradson Carbon and 0-500 ppm Ni & V. The amount of metals depends on the type of feed and other processes that have been run on the feedstock before processing the feed with the composition of this invention.

The catalyst may also be used in fixed bed and moving bed catalytic cracking processes. The catalyst for these processes is generally in extrudate or pellet form, and those catalysts typically have parameters on the magnitude of 0.5 to 1.5 mm in diameter to 2-5 mm in length.

The amount of olefins produced and the ratios of specific olefins produced will depend on a number of factors, including but not limited to, the type and metals content of the feed being processed, the cracking temperature, the amount of olefins producing additives used, and the type of cracking unit, e.g., FCC versus a deep catalytic cracking (DCC) unit. Based on data on cracked products from a Davison Circulating Riser, the anticipated cracked product stream obtained, using these preferred catalysts, will typically contain from 8 to 40 wt. % C₃ and C₄ olefins.

The invention can also be used in areas outside of catalytic cracking, especially those compositions of the invention comprising non-zeolitic sieves that are typically used in purification processes. The composition for those applications may also be in the form of particulates, extrudates and/or pellets.

Having described the basic aspects of the invention, the following specific examples are given merely to illustrate the preferred embodiments of the invention and are not intended to a limit in any way the claims appended hereto.

### EXAMPLES

### Example 1

### Preparation of a Ferric Phosphate Bound Zeolite

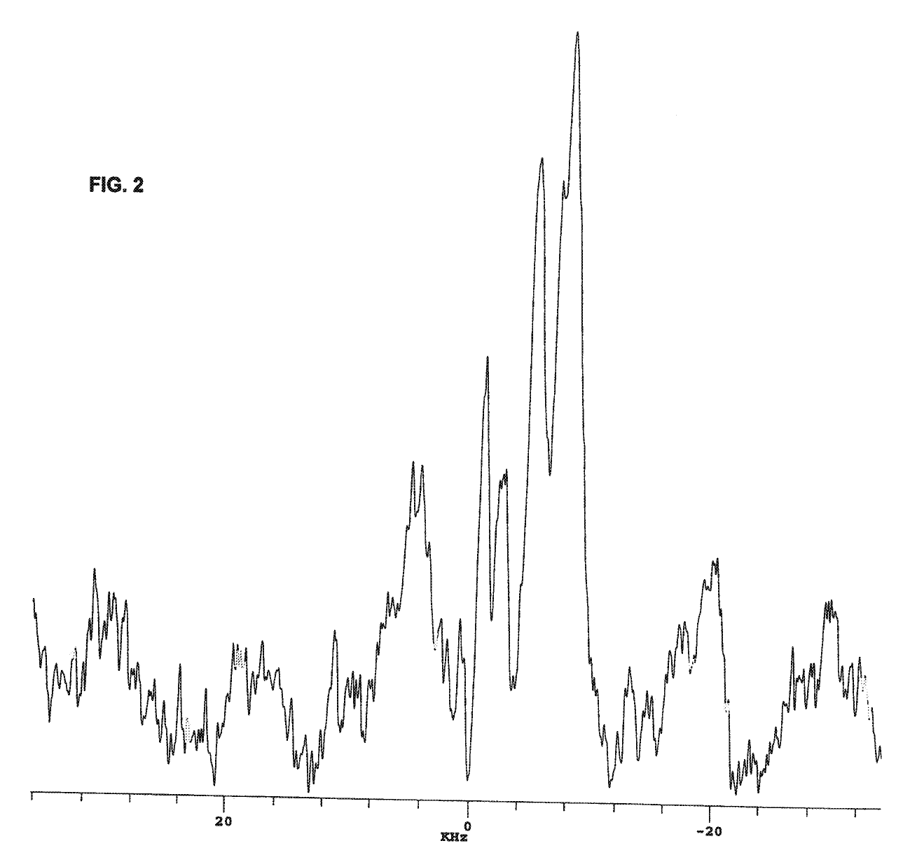

1690 g of FeCl₃.6H₂O was dissolved in 7000 g H₂O. To this aqueous solution was added 2000 g ZSM-5 (the amount of ZSM 5 in this Example and the amounts reported in the Examples that follow being reported on a dry basis). The resulting slurry was mixed and heated to 80° C. for one hour. 856 g of phosphoric acid was then added and stirred. 1880 g of kaolin clay (the amount of clay in this Example and the amounts reported in the Examples that follow being reported on a dry basis) was added to the slurry and mixed for five minutes prior to milling the slurry. The slurry was milled in a Drais mill. The pH of the slurry was 0.03. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample prepared according to this Example 1 was also subjected to nuclear magnetic resonance analysis to confirm the formation of the metal phosphate. The results appear in FIG. 2. The conditions for running the NMR for this sample and those described herein are as follows. The ³¹P nuclear magnetic resonance (NMR) experiments were performed on a Chemagnetics Infinity 400 MHz solid-state spectrometer (magnetic field 9.4T) operating at a resonance frequency of 161.825 MHz. A 4 mm Chemagnetics pencil probe was utilized to acquire all of the data. Samples were spun at 12 kHz. Samples were referenced to an external 85% H₃PO₄ solution. All data was acquired using a bloch decay sequence. A pulse length of 4 μs and a recycle delay of 30 seconds were utilized for all samples. One hundred twenty eight (128) acquisitions were performed on all samples except FePO₄ in this Example 1 for which 8000 acquisitions were performed. Fourier Transformation was applied to all time data to obtain the displayed spectra.

### Example 2

### Preparation of a Calcium Phosphate Bound Zeolite

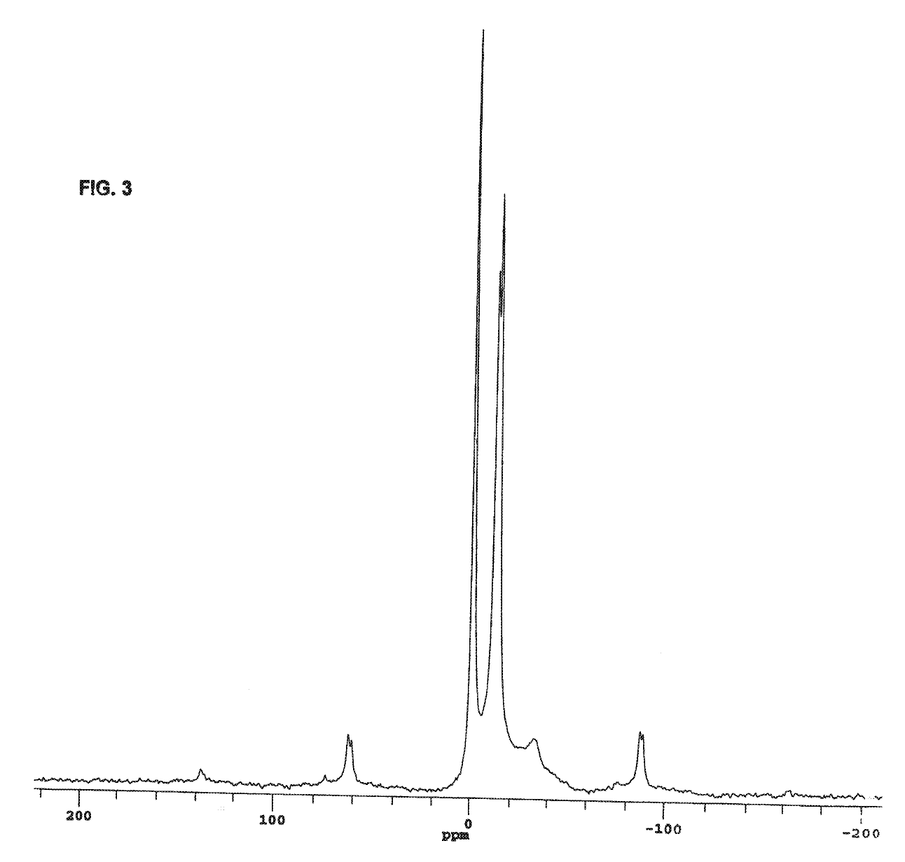

1180 g of CaCl₂.2H₂O was dissolved in 5800 g of H₂O. To this aqueous solution was added 1800 g ZSM-5. The resulting slurry was mixed and heated to 80° C. for one hour. 807 g of phosphoric acid was then added and stirred. 1666 g of clay was added to the slurry and mixed for five minutes prior to milling the slurry. The slurry was milled. The pH of the slurry was 0.55. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 3.

### Example 3

### Preparation of a Calcium Phosphate Bound Zeolite (12% Phosphoric Acid)

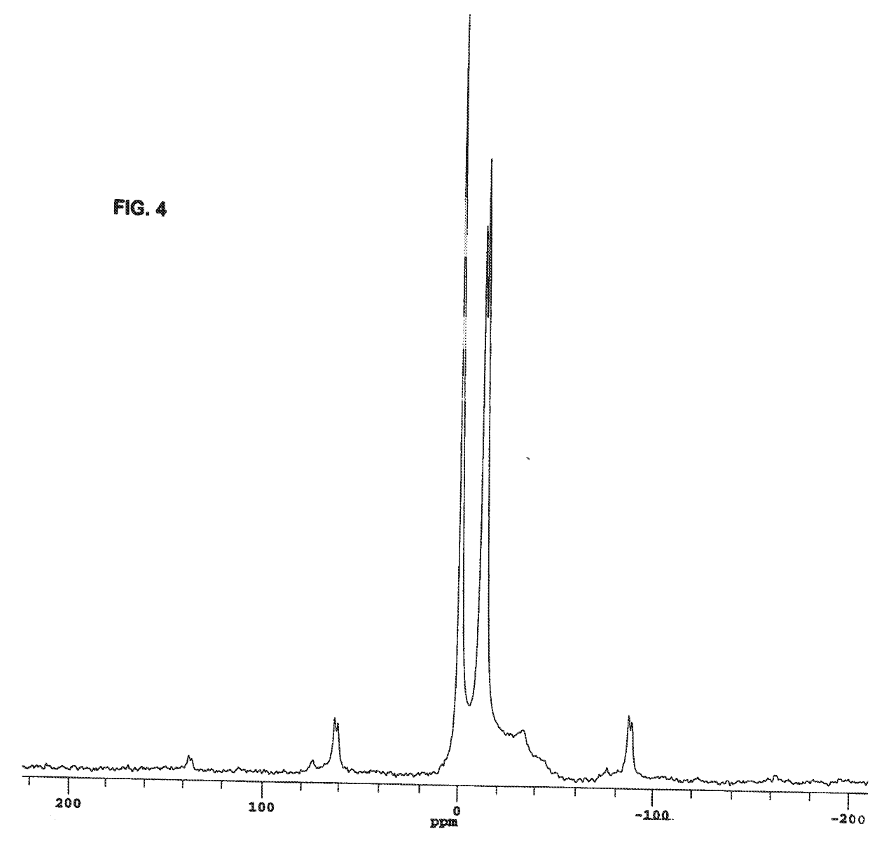

Example 2 was repeated, but with a slightly less concentrated phosphoric acid solution. More particularly, 1311 g of CaCl₂.2H₂O was dissolved in 7000 g H₂O. To this solution was added 2000 g ZSM-5. The resulting slurry was mixed and heated to 80° C. for one hour. 828 g of phosphoric acid was then added and stirred. 1900 g of clay was added to the slurry and mixed for five minutes prior to milling the slurry. The slurry was milled. The pH of the slurry was 0.10. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 4.

### Example 4

### Preparation of a Calcium Phosphate Bound Zeolite (7.7% Phosphoric Acid)

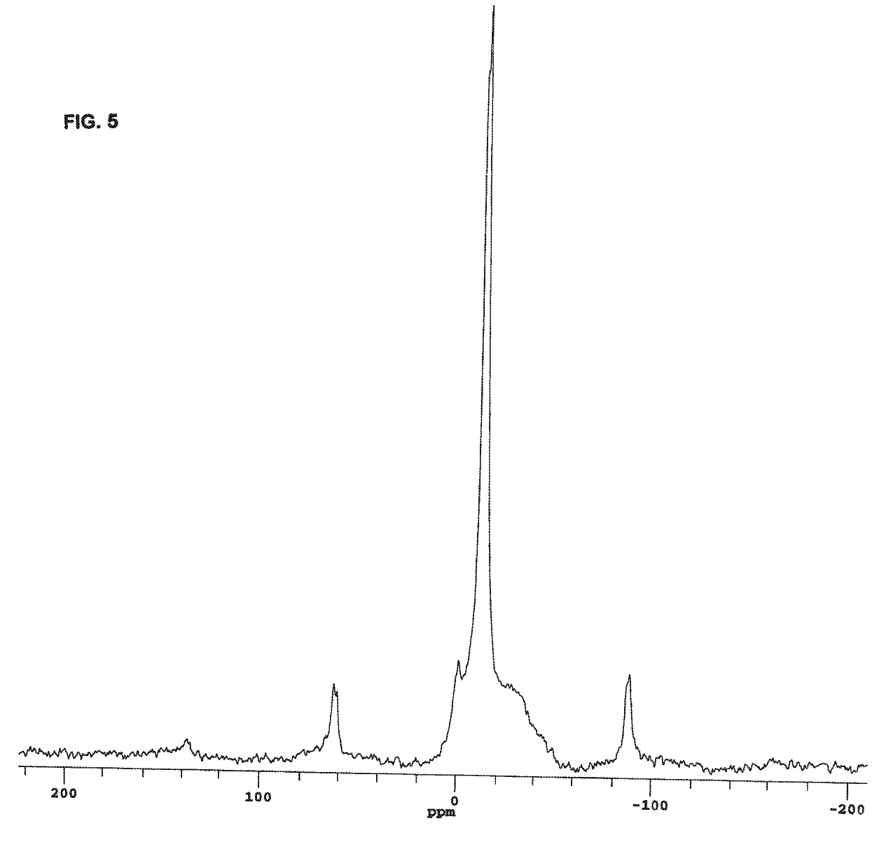

Example 2 was repeated except the concentration of phosphoric acid was significantly reduced to 7.7%. More particularly, 656 g of CaCl₂.H₂O was dissolved in 6268 g H₂O. To this solution was added 2000 g ZSM-5. The resulting slurry was mixed and heated to 80° C. for one hour. 531 g of phosphoric acid was then added and stirred. 2365 g of clay was added to the slurry and mixed for five minutes prior to milling the slurry. The slurry was milled. The pH of the slurry was 1.41. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 5.

### Example 5 (Comparison)

### Preparation of a Aluminum Phosphate Bound Zeolite

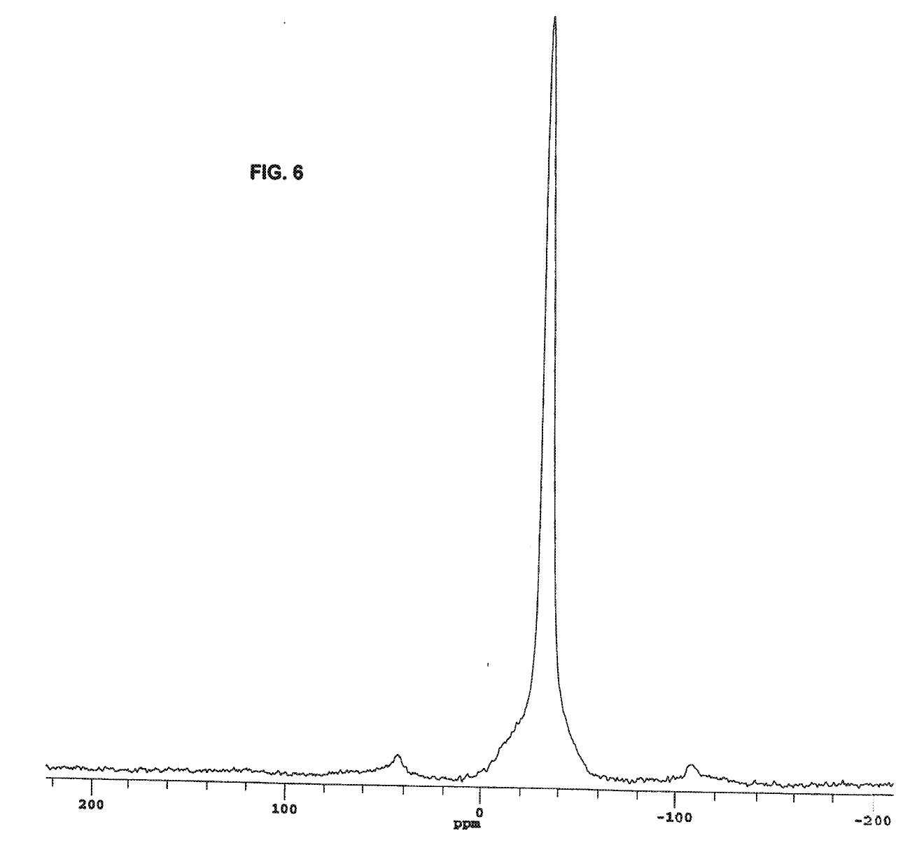

1184 g of AlCl₃.6H₂O was dissolved in 5676 g H₂O. To this solution was added 2000 g ZSM-5. The resulting slurry was mixed and heated to 80° C. for one hour. 725 g of phosphoric acid was then added and stirred. 2225 g of clay was added to the slurry and mixed for five minutes prior to milling the slurry. The slurry was milled. The pH of the slurry was 1.24. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 6

### Example 6

### Preparation of a Strontium Phosphate Bound Zeolite

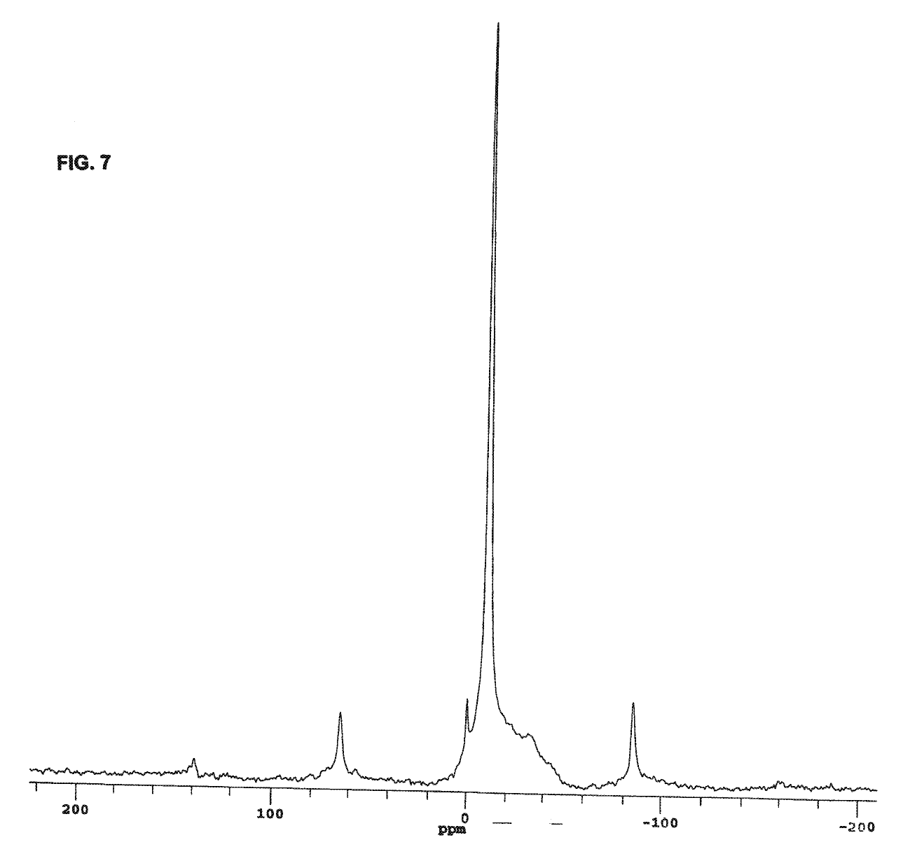

1072 g of SrCl₂.6H₂O was dissolved in 5800 g of H₂O. To this solution was added 1666 g ZSM-5. The resulting slurry was mixed and heated to 80° C. for one hour. 1166 g of phosphoric acid was then added and stirred. 1746 g of clay was added to the slurry and mixed for five minutes prior to milling the slurry. The slurry was milled. The pH of the slurry was 0.26. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 7.

### Example 7

### Preparation of a Lanthanum Phosphate Bound Zeolite

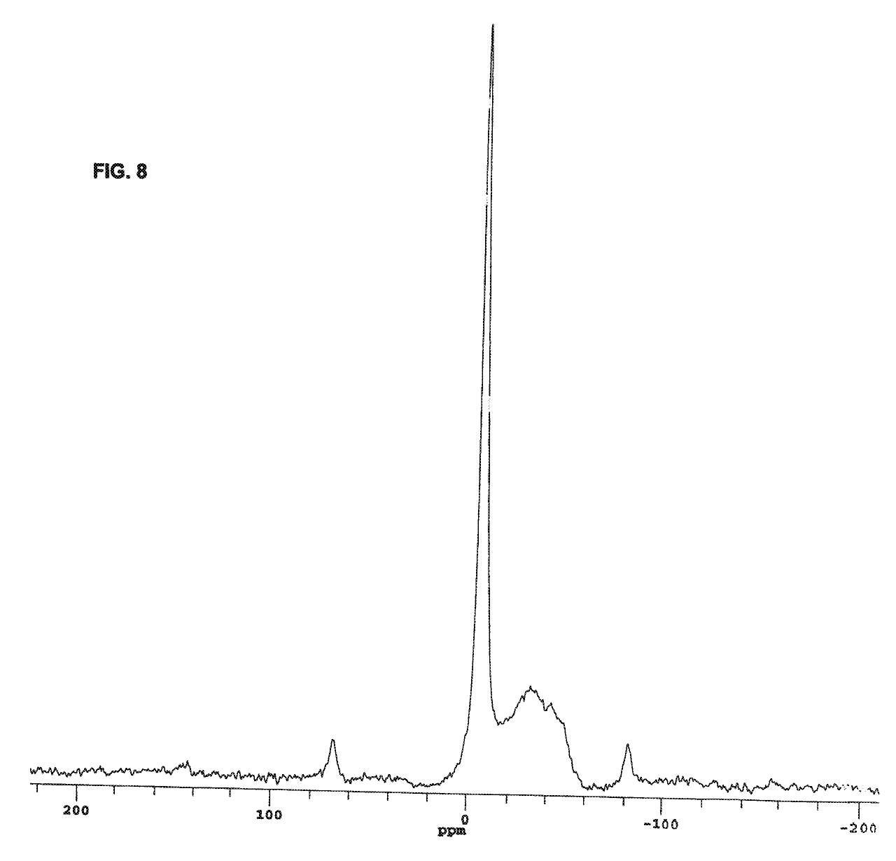

1140 g of LaCl₃.6H₂O was dissolved in 7000 g of H₂O. To this solution was added 2000 g ZSM-5. The resulting slurry was mixed and heated to 80° C. for one hour. 545 g of phosphoric acid was then added and stirred. 2105 g of clay was added to the slurry and mixed for five minutes prior to milling the slurry. The slurry was milled. The pH of the slurry was 0.18. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 8.

### Example 8

### Preparation of a Magnesium Phosphate Bound Zeolite

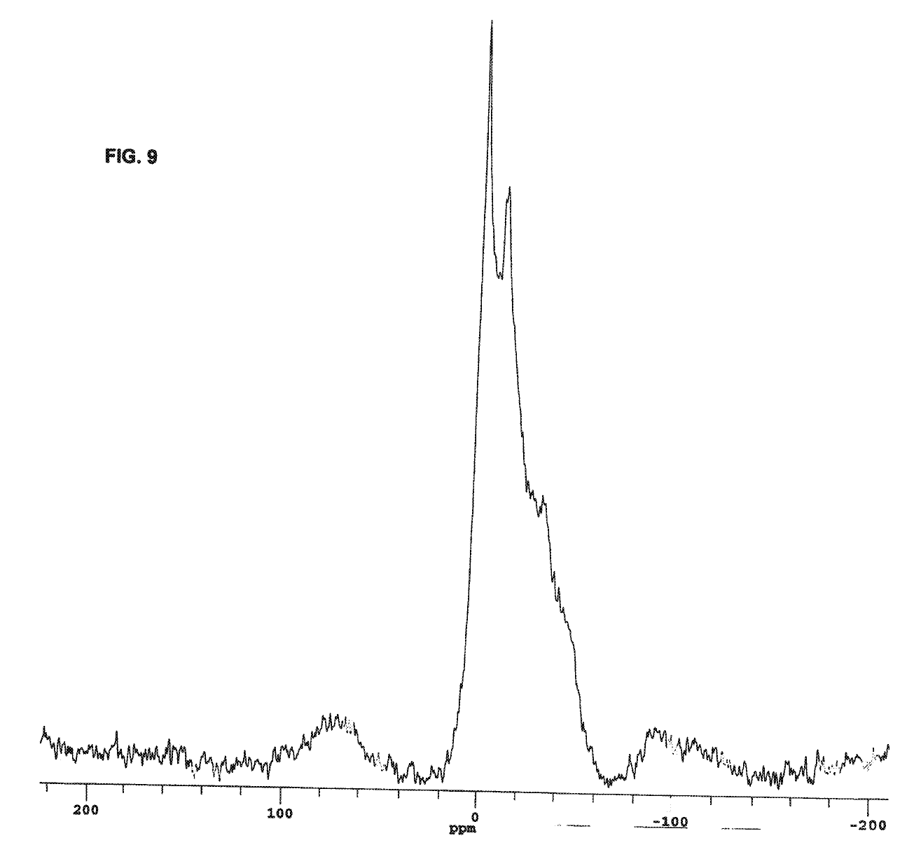

1261 g of MgCl₂.6H₂O was dissolved in 5625 g of H₂O. To this solution was added 2000 g ZSM-5. The resulting slurry was mixed and heated to 80° C. for one hour. 649 g of phosphoric acid was then added and stirred. 2280 g of clay was added to the slurry and mixed for five minutes prior to milling the slurry. The slurry was milled. The pH of the slurry was 1.22. The resulting milled slurry was then spray dried at an inlet temperature and outlet temperature of 399° C. and 149° C., respectively to form particles having a mean particle size reported in Table 1. The spray dried catalyst particles were then calcined for forty minutes at 593° C. in a lab muffle. The content of the catalyst prepared in this example and various properties of the catalyst, such as average (mean) particle size, average bulk density, etc., are provided in Table 1 below. The sample was also subjected to NMR analysis according to conditions described in Example 1. The results appear in FIG. 9.

### Example 9

### Olefin Yields Obtained Using the Invention

Each of the catalysts prepared in Examples 1-8, and two commercially available catalysts, were tested for olefin production in a Davison Circulating Riser that is designed to simulate the conditions of a conventional FCC unit. The description and operation of the DCR has been published in the following papers: G. W. Young, G. D. Weatherbee, and S. W. Davey, “Simulating Commercial FCCU Yields With The Davison Circulating Riser (DCR) Pilot Plant Unit,” National Petroleum Refiners Association (NPRA) Paper AM88-52; G. W. Young, “Realistic Assessment of FCC Catalyst Performance in the Laboratory,” in Fluid Catalytic Cracking: Science and Technology, J. S. Magee and M. M. Mitchell, Jr. Eds. Studies in Surface Science and Catalysis Volume 76, p. 257, Elsevier Science Publishers B.V., Amsterdam 1993, ISBN 0-444-89037-8.

The inventive catalysts were tested with conventional faujasite-based catalyst, i.e., Aurora 168 LLIM catalyst. Each of the catalysts described in Examples 1-8 were blended with the aforementioned Aurora product at a level of 8% by weight. These blends were compared against the same Aurora product without the invention, as well as compared against the Aurora product containing 8% by weight of OlefinsUltra™ catalyst, an olefins catalyst commercially available from W.R. Grace & Co.—Conn. All of the catalysts were steamed in a fluidized bed for 4 hours at 816° C. under 100% steam atmosphere before evaluation. The reactor/stripper temperature of the DCR was 521° C. The regenerator was operated at 704° C. and full burn with 1% excess O₂. The feed was heated between 149° C. and 371° C. to obtain different conversions. The feed used had properties indicated in Table 2 below. The octane number results are generated using G-Comm analysis, which has been described in “Fluid Catalytic Cracking”: Science and Technology, Vol. 76, p. 279, Ed. Mageland Mitchell.

The interpolated results of the DCR testing are provided in Table 3 below. The parameters marked with the double asterisks (**) are those used to measure the performance of the catalysts relative to light olefins production. It is shown that the catalyst compositions of this invention provide additional compositions for making olefins and in at least one embodiment (Example 1), provides a catalyst having enhanced production compared to standard catalyst (Aurora), a commercially available olefins catalyst (Olefins Ultra) and an aluminum phosphate bound catalyst made according to U.S. Pat. No. 5,194,412 (Example 5).

The RON results below also indicate that a refiner can use the invention to manipulate and/or enhance olefin yields and at the same time produce higher octane gasoline, albeit at lower gasoline yields.

Table 3 below also includes a complete listing of yields of other products from cracking the hydrocarbon feedstream. The yields reported were obtained using gas chromatography.

### Example 10

### Preparation of Yttrium Phosphate Bound Zeolite

1471 grams of yttrium rich rare earth chloride solution (19.14% Y₂O₃ and 20.4% RE₂O₃) was mixed with 5432 grams of H₂O. To this solution 503 grams of H₃PO₄ solution was added and mixed. Then 1250 grams of ZSM-5 were added to the slurry and mixed. Then 3140 grams of clay was added and mixed. The slurry was milled in a Drais mill operated in conditions described in Example 1. The pH of the slurry was zero. The milled slurry was then spray dried at inlet temperature and outlet temperature of 399° C. and 149° C., respectively, to form particles having a mean particle size reported in the Table 4. The spray dried particles were then calcined for forty minutes at 593° C. in a lab muffle. Properties of this sample are shown in Table 4.

### Example 11

### Preparation of Lanthanum Phosphate Bound Zeolite

1098 grams of lanthanum rich rare earth chloride solution (27.22% La₂O₃ and 27.31% RE₂O₃) was mixed with 5831 grams of H₂O. To this solution 406 grams of H₃PO₄ solution was added and mixed. Then 1250 grams of ZSM-5 were added to the slurry and mixed. Then 3200 grams of clay was added and mixed. The slurry was milled in a Drais mill operated in conditions described in Example 1. The pH of the slurry was 0.72. The milled slurry was then spray dried at inlet temperature and outlet temperature of 399° C. and 149° C., respectively, to form particles having a mean particle size reported in the Table 4. The spray dried particles were then calcined for forty minutes at 593° C. in a lab muffle. Properties of this sample are shown in Table 4.

### Example 12

### C₃/C₄ Selectivity of Yttrium Phosphate Bound Zeolite

The catalysts prepared in Examples 10 and 11 were tested for olefin production in a Davison Circulating Riser that was operated according to the same conditions described in Example 9 using the same feed. Prior to testing, the catalysts from Example 10 and Example 11 were steamed for twenty four hours at 816° C., with 100% steam. The catalysts in Examples 10 and 11 were then tested at 5% by weight of the catalyst, compared to 8% in the early examples with the remaining portion of the catalyst being Aurora 168LLIM. As shown in Table 5, the yttrium phosphate bound zeolite of Example 10 showed to be more selective for butylene than Example as shown by the lower C₃=/C₄=ratio, while maintaining substantially the same yields for other olefins save for having a lower C₃=yield compared to Example 11.

TABLE 1

| | | EXAMPLE | | | | | | | |
|---|---|---|---|---|---|---|---|---|---|
| | | 1 | 2 | 3 | 4 | 5 | 6 | 7 | 8 |
| | | 40% ZSM-5 | 40% ZSM-5 | 40% ZSM-5 | 40% ZSM-5 | 40% ZSM-5 | 40% ZSM-5 | 40% ZSM-5 | 40% ZSM-5 |
| | | 10% Fe₂O₃ | 10% CaO | 10% CaO | 5% CaO | 5% Al₂O₃ | 10% SrO | 10% La₂O₃ | 5% MgO |
| | | (FeCl₃) 1 Hr. | (CaCl₂) 1 | (CaCl₂) | (CaCl₂) | (AlCl₃) 1 Hr. | (SrCl₂) 1 Hr. | (LaCl₃) | (MgCl₂) |
| | | @ 80° C. | Hr. @ 80° C. | 1 Hr. @ 80° C. | 1 Hr. @ 80° C. | @ 80° C. | @ 80° C. | 1 Hr. @ 80° C. | 1 Hr. @ 80° C. |
| | | 12.4% P₂O₅ | 13% P₂O₅ | 12% P₂O₅ | 7.7% P₂O₅ | 10.5% P₂O₅ | 8.1% P₂O₅ | 7.9% P₂O₅ | 9.4% P₂O₅ |
| | Comparison | (H₃PO₄) | (H₃PO₄) | (H₃PO₄) | (H₃PO₄) | (H₃PO₄) | (H₃PO₄) | (H₃PO₄) | (H₃PO₄) |
| | OlefinsUltra¹ | 37.6% Clay² | 37% Clay | 38% Clay | 47.3% Clay | 44.5% Clay | 41.9% Clay | 42.1% Clay | 45.6% Clay |
| Al₂O₃ | 27 | 18.1 | 18.4 | 18.4 | 22 | 26.2 | 20.1 | 20.3 | 21.9 |
| Na₂O | 0.17 | 0.11 | 0.14 | 0.13 | 0.1 | 0.1 | 0.13 | 0.11 | 0.12 |
| MgO | 0.06 | 0.06 | 0.07 | 0.06 | 0.06 | 0.36 | 0.06 | 0.06 | 4.56 |
| CaO | 0.07 | 0.11 | 8.59 | 8.64 | 4.84 | 0.14 | 0.11 | 0.11 | 0.54 |
| SrO₃ | | | | | | | 9.28 | | |
| Fe₂O₃ | 0.59 | 10.42 | 0.56 | 0.6 | 0.71 | 0.71 | 0.67 | 1.19 | 0.72 |
| La₂O₃ | 0.03 | 0.03 | 0.01 | 0.01 | 0.01 | 0.01 | 0.02 | 9.19 | 0.01 |
| P₂O₅ | 11.6 | 13.33 | 13.29 | 13.01 | 7.69 | 10.24 | 8.92 | 8.99 | 9.26 |
| APS⁴ | 71 | 66 | 81 | 77 | 74 | 65 | 69 | 66 | 64 |
| ABD⁵ | 0.69 | 0.73 | 0.64 | 0.63 | 0.66 | 0.7 | 0.67 | 0.66 | 0.71 |
| DI⁶⁶ | 8 | 10 | 2 | 3 | 3 | 7 | 12 | 5 | 9 |
| ZSA⁷ | 122 | 113 | 131 | 119 | 121 | 125 | 121 | 121 | 125 |
| MSA⁸ | 24 | 17 | 23 | 34 | 32 | 19 | 30 | 44 | 22 |
| TSA⁹ | 166 | 130 | 154 | 153 | 153 | 144 | 151 | 165 | 147 |

4 Hrs. @ 816° C. Steam

| | | | | | | | | | |
|---|---|---|---|---|---|---|---|---|---|
| TSA | 150 | 131 | 132 | 128 | 124 | 137 | 114 | 145 | 89 |

¹Olefins Ultra ™ additive does not contain a metal phosphate as defined herein and is commercially available from W.R. Grace &Co.-Conn.
²Natka clay
³Strontium oxide was only measured for the sample from Example 6.
⁴APS = mean particle size as measured by Malvern Mastersizer-S.
⁵ABD = average bulk density
⁶Davison Attrition Index measured as described earlier
⁷zeolite surface area that is determined by t-plot.
⁸matrix surface area as measured by t-plot.
⁹total surface area as measured by BET.

TABLE 2
Simulated Distillation, Vol. % ° F.:

| | | | | | |
|---|---|---|---|---|---|
| API Gravity @ 60° F. | 25.5 | Al ppm: | 0 | IBP: | 307 |
| Specific Gravity @ 60° F. | 0.9012 | Ca ppm: | 0 | 5 | 513 |
| Aniline Point, ° F. | 196 | Mg ppm: | 0 | 10 | 607 |
| Sulfur, Wt. % | 0.396 | Zn ppm: | 0 | 20 | 691 |
| Total Nitrogen, Wt. % | 0.12 | P ppm: | 0 | 30 | 740 |
| Basic Nitrogen, Wt. % | 0.05 | Pb ppm: | 0 | 40 | 782 |
| Conradson Carbon, Wt. % | 0.68 | Cr ppm: | 0 | 50 | 818 |
| Ni, ppm | 0.4 | Mn ppm: | 0 | 60 | 859 |
| V, ppm | 0.2 | Sb ppm: | 0 | 70 | 904 |
| Fe, ppm | 4 | Ba ppm: | 0.1 | 80 | 959 |
| Cu, ppm | 0 | K ppm: | 0 | 90 | 1034 |
| Na, ppm | 1.2 | | | 95 | 1103 |
| Refractive Index | 1.5026 | | | FPB | 1257 |
| Average Molecular Weight | 406 | | | PCT | 99.3 |
| % Aromatic Ring | 18.9 | | | | |
| Carbons, Ca | | | | | |
| % Paraffinic Carbons, Cp | 63.6 | | | | |
| Naphthenic Carbons, Cn | 17.4 | | | | |
| K Factor | 11.94 | | | | |

TABLE 3

| | Comparison | Comparison | | | | | | | | |
|---|---|---|---|---|---|---|---|---|---|---|
| | #1 | #2 | Exam- | Exam- | | | | | | |
| Catalyst | Aurora ™- | Olefins | ple 1 | ple 2 | Example 3 | Example 4 | Example 5 | Example 6 | Example 7 | Example 8 |
| Composition | 168LLIM¹¹ | Ultra ™ | Ex. 1 | Ex. 2 | Ex. 3 | Ex. 4 | Ex. 5 | Ex. 6 | Ex. 7 | Ex. 8 |
| (% by weight)¹⁰ | (100%) | (8%) | (8%) | (8%) | (8%) | (8%) | (8%) | (8%) | (8%) | (8%) |
| Conversion | 70 | 70 | 70 | 70 | 70 | 70 | 70 | 70 | 70 | 70 |
| Activity | 7.07 | 7.62 | 7.32 | 7.79 | 7.11 | 7.64 | 7.48 | 7.39 | 7.08 | 7.29 |
| H₂ Yield wt % | 0.03 | 0.03 | 0.05 | 0.03 | 0.04 | 0.03 | 0.03 | 0.03 | 0.05 | 0.03 |
| C₁ + C₂'s wt % | 2.07 | 2.18 | 2.23 | 2.04 | 2.11 | 2.03 | 2.04 | 1.99 | 2.05 | 1.98 |
| C₂ wt % | 0.63 | 0.52 | 0.47 | 0.52 | 0.55 | 0.53 | 0.51 | 0.52 | 0.51 | 0.54 |
| **C₂= wt % | 0.69 | 0.99 | 1.14 | 0.85 | 0.85 | 0.81 | 0.87 | 0.78 | 0.88 | 0.75 |
| Total C₃ wt % | 4.87 | 9.56 | 10.65 | 8.75 | 8.31 | 8.24 | 8.67 | 7.91 | 9.03 | 7.25 |
| **C₃= wt % | 4.25 | 8.75 | 9.80 | 8.01 | 7.57 | 7.52 | 7.92 | 7.20 | 8.29 | 6.57 |
| Total C₄ wt % | 9.14 | 12.82 | 13.28 | 12.93 | 12.58 | 12.51 | 12.40 | 12.09 | 12.92 | 11.87 |
| iC₄ wt % | 1.81 | 2.37 | 2.35 | 2.23 | 2.29 | 2.20 | 2.27 | 2.26 | 2.29 | 2.16 |
| nC₄ wt % | 0.41 | 0.49 | 0.50 | 0.46 | 0.47 | 0.45 | 0.47 | 0.46 | 0.47 | 0.45 |
| **Total C₄= | 6.90 | 9.98 | 10.55 | 10.30 | 9.74 | 9.83 | 9.77 | 9.56 | 10.26 | 9.23 |
| wt % | | | | | | | | | | |
| C₄= wt % | 1.36 | 1.84 | 1.93 | 1.87 | 1.78 | 1.80 | 1.78 | 1.77 | 1.84 | 1.69 |
| iC₄= wt % | 2.39 | 3.89 | 4.09 | 3.97 | 3.67 | 3.76 | 3.73 | 3.64 | 3.95 | 3.45 |
| tC₄= wt % | 1.75 | 2.39 | 2.56 | 2.51 | 2.44 | 2.40 | 2.40 | 2.35 | 2.56 | 2.32 |
| cC₄= wt % | 1.32 | 1.81 | 1.92 | 1.90 | 1.79 | 1.82 | 1.80 | 1.75 | 1.85 | 1.70 |
| Gasoline wt % | 51.76 | 43.00 | 40.84 | 43.80 | 45.09 | 44.90 | 44.41 | 45.54 | 43.62 | 46.62 |
| G-Con P wt % | 3.44 | 3.47 | 3.57 | 3.39 | 3.38 | 3.39 | 3.47 | 3.39 | 3.49 | 3.36 |
| G-Con I wt % | 20.07 | 16.24 | 15.45 | 16.23 | 17.07 | 17.03 | 16.88 | 17.51 | 16.88 | 18.07 |
| G-Con A wt % | 29.99 | 34.04 | 35.45 | 32.30 | 32.65 | 32.12 | 33.10 | 32.34 | 33.16 | 32.00 |
| G-Con N wt % | 11.98 | 10.11 | 10.12 | 10.00 | 10.15 | 10.36 | 10.62 | 10.51 | 10.35 | 10.94 |
| G-Con O wt % | 34.94 | 36.36 | 36.03 | 38.58 | 37.71 | 37.53 | 36.75 | 36.63 | 36.54 | 36.59 |
| **G-Con | 92.19 | 94.09 | 94.21 | 94.08 | 93.89 | 93.77 | 93.66 | 93.67 | 93.65 | 93.31 |
| RON EST | | | | | | | | | | |
| **G-Con | 78.56 | 79.75 | 79.87 | 79.62 | 79.56 | 79.45 | 79.54 | 79.36 | 79.41 | 79.24 |
| MON EST | | | | | | | | | | |
| LCO wt % | 22.29 | 21.66 | 21.53 | 21.61 | 21.49 | 21.70 | 21.76 | 21.56 | 21.96 | 21.86 |
| Bottoms wt % | 7.71 | 8.34 | 8.47 | 8.39 | 8.51 | 8.30 | 8.24 | 8.44 | 8.04 | 8.14 |
| Coke wt % | 2.21 | 2.42 | 2.59 | 2.32 | 2.32 | 2.32 | 2.31 | 2.31 | 2.34 | 2.40 |
| **C₃=/C₄= | 0.62 | 0.88 | 0.93 | 0.78 | 0.78 | 0.77 | 0.81 | 0.75 | 0.81 | 0.71 |

¹⁰Indicates the amount of component listed, based on total catalyst composition. The first comparison example comprises 100% Aurora 168LLIM catalyst. For the remaining examples OlefinsUltra catalyst and catalysts from Examples 1-8 were each separately blended with Aurora catalyst in an amount of 8% by weight of the total composition, and the remaining 92% being the aforementioned Aurora catalyst.
¹¹Aurora ™ 1168LLIM catalyst does not contain metal phosphate binder as described herein and is commercially available from W. R. Grace & Co.-Conn.

TABLE 4

EXAMPLE

| | 10 | 11 |
|---|---|---|
| | 25% (by wt.) | 25% (by wt.) |
| | ZSM-5 | ZSM-5 |
| | 5% P₂O₅ | 6.2% P₂O₅ |
| | 6% La₂O₃ | 6% Y₂O₃ |
| | 64% Clay¹ | 62.8% Clay¹ |
| Al₂O₃ | 29 | 27.9 |
| Na₂O | 0.14 | 0.14 |
| P₂O₅ | 5.86 | 6.92 |
| Y₂O₃ | 0.003 | 6.02 |
| La₂O₃ | 6.06 | 0.21 |
| RE₂O₃ | 6.09 | 6.49 |
| APS² | 50 | 65 |
| ABD³ | 0.72 | 0.74 |
| DI⁴ | 9 | 17 |
| TSA⁵ | 108 | 108 |
| ZSA⁶ | 74 | 81 |
| MSA⁷ | 34 | 27 |

24 Hrs. @ 816° C., 100% Steam

| | | |
|---|---|---|
| TSA | 96 | 90 |
| ZSA | 62 | 54 |
| MSA | 34 | 36 |

¹Natka Clay
²APS = mean particle size as measured by Malvern Mastersizer-S
³ABD = average bulk density
⁴Davison Attrition Index measured as described earlier
⁵total surface area as measured by BET
⁶zeolite surface area that is determined ty t-plot
⁷matrix surface area as measured by t-plot

TABLE 5

| Catalyst Composition | Ex. 10* | Ex. 11* |
|---|---|---|
| (% by weight) | (5%) | (5%) |
| Conversion | 74 | 74 |
| Activity | 8.55 | 8.36 |
| H₂ Yield wt % | 0.03 | 0.03 |
| C₁ + C₂'s wt % | 2.01 | 1.97 |
| C₂ wt % | 0.57 | 0.55 |
| C₂ = wt % | 0.71 | 0.70 |
| Total C₃ wt % | 6.55 | 6.90 |
| C₃ = wt % | 5.86 | 6.20 |
| Total C₄ wt % | 11.26 | 11.45 |
| iC₄ wt % | 2.58 | 2.63 |
| nC₄ wt % | 0.49 | 0.49 |
| Total C₄ = wt % | 8.38 | 8.50 |
| C₄ = wt % | 1.65 | 1.66 |
| iC₄ = wt % | 2.95 | 3.05 |
| tC₄ = wt % | 2.14 | 2.15 |
| cC₄ = wt % | 1.59 | 1.59 |
| Gasoline wt % | 50.88 | 50.63 |
| G-Con P wt % | 3.38 | 3.33 |
| G-Con I wt % | 21.65 | 21.32 |
| G-Con A wt % | 28.75 | 29.06 |
| G-Con N wt % | 10.39 | 10.11 |
| G-Con O wt % | 35.74 | 36.10 |
| G-Con RON EST | 93.15 | 93.46 |
| G-Con MON EST | 79.67 | 79.85 |
| LCO wt % | 19.49 | 19.64 |
| Bottoms wt % | 6.36 | 6.25 |
| Coke wt % | 2.96 | 2.93 |
| C₃=/C₄= | 0.70 | 0.73 |

*Additives steamed for 24 Hrs. @ 816° C., 100% steam. The weight percent in parentheses indicates the amount of the additive from the example included in the total catalyst composition with the remainder of the catalyst composition being the aforementioned Aurora 1168LLIM catalyst that does not contain metal phosphate binder as described herein.

What is claimed:

1. A catalyst composition comprising
(a) zeolite,
(b) aluminum phosphate, and
(c) metal phosphate present in an amount sufficient for the metal phosphate to at least function as a binder for the zeolite and the metal is other than aluminum.

2. A catalyst composition according to claim 1 wherein the metal of (c) is selected from the group consisting of Group IIA metals, lanthanide series metals, scandium, yttrium, lanthanum, and transition metals.

3. A catalyst composition according to claim 1 wherein the metal of (c) is selected from the group consisting of iron, lanthanum and calcium.

4. A catalyst composition according to claim 1 comprising at least 5% by weight of the metal phosphate as measured by amount of the metal's corresponding oxide present in the composition.

5. A catalyst composition according to claim 1 comprising about 4% to about 50% by weight of the metal phosphate as measured by amount of the metal's corresponding oxide present in the composition.

6. A catalyst composition according to claim 5 further comprising a member of the group consisting of clay, silica, alumina, silica-alumina, yttria, lanthana, ceria, neodymia, samaria, europia, gadolinia, titania, zirconia, praseodymia and mixtures thereof.

7. A catalyst composition according to claim 1 wherein zeolite (a) is selected from ZSM-5, beta zeolite, mordenite, ferrierite and any other zeolite having a silica to alumina molar ratio of twelve or greater.

8. A catalyst according to claim 1 wherein the zeolite is ZSM-5.

9. A catalyst according to claim 2 wherein the zeolite is ZSM-5.

10. A catalyst according to claim 3 wherein the zeolite is ZSM-5.

11. A catalyst according to claim 4 wherein the zeolite is ZSM-5.

12. A catalyst according to claim 5 wherein the zeolite is ZSM-5.

13. A catalyst according to claim 6 wherein the zeolite is ZSM-5.

14. A catalyst composition according to claim 1 wherein the composition is particulated and fluidizable.

15. A catalyst composition according to claim 14 wherein the catalyst has a mean particle size in the range of 20 to 150 microns.

16. A catalyst composition according to claim 1 wherein the composition is in the form of an extrudate or pellet.

17. A catalyst composition according to claim 1 wherein the composition has a Davison Attrition Index in the range of 0 to about 30.

18. A catalyst composition according to claim 1 wherein the composition has a Davison Attrition Index in the range of 0 to about 20.

19. A catalyst composition comprising
(a) zeolite,
(b) metal phosphate present in an amount sufficient for the metal phosphate to at least function as a binder for the zeolite and the metal is other than aluminum, wherein the metal phosphate comprises at least 5% by weight of the catalyst composition as measured by amount of the metal's corresponding oxide.

20. A catalyst composition according to claim 19 wherein the metal is selected from the group consisting of Group IIA metals, lanthanide series metals, scandium, yttrium, lanthanum, and transition metals.

21. A catalyst composition according to claim 19 wherein the metal is selected from the group consisting of iron, lanthanum and calcium.

22. A catalyst composition according to claim 19 further comprising a member of the group consisting of clay, silica, alumina, silica-alumina, yttria, lanthana, ceria, neodymia, samaria, europia, gadolinia, titania, zirconia, praseodymia and mixtures thereof.

23. A catalyst composition according to claim 19 wherein the zeolite is selected from ZSM-5, mordenite, ferrierite and any other zeolite having a silica to alumina molar ratio of twelve or greater.

24. A catalyst according to claim 19 wherein the zeolite is ZSM-5.

25. A catalyst according to claim 20 wherein the zeolite is ZSM-5.

26. A catalyst according to claim 21 wherein the zeolite is ZSM-5.

27. A catalyst according to claim 22 wherein the zeolite is ZSM-5.

28. A catalyst composition according to claim 19 comprising about 4% to about 50% by weight of the metal phosphate as measured by amount of the metal's corresponding oxide present in the composition.

29. A catalyst according to claim 28 wherein the zeolite is ZSM-5.

30. A catalyst composition according to claim 19 wherein the composition is particulated and fluidizable.

31. A catalyst composition according to claim 30 wherein the catalyst has a mean particle size in the range of 40 to 150 microns.

32. A catalyst composition according to claim 19 wherein the composition has a Davison Attrition Index in the range of 0 to about 30.

33. A catalyst composition according to claim 19 wherein the composition has a Davison Attrition Index in the range of 0 to about 30.

34. A method for catalytic cracking of hydrocarbons that comprises reacting a hydrocarbon under catalytic cracking conditions in the presence of a catalyst comprising
(a) zeolite,
(b) aluminum phosphate,
(c) metal phosphate present in an amount sufficient for it to at least function as a binder for the zeolite and the metal is other than aluminum.

35. A method according to claim 34 wherein the metal of (c) is selected from the group consisting of Group IIA metals, lanthanide series and Group VIII metals.

36. A method according to claim 34 wherein the metal of (c) is selected from the group consisting of iron, lanthanum and calcium.

37. A method according to claim 34 wherein the catalyst comprises at least 5% by weight of the metal phosphate as measured by amount of the metal's corresponding oxide present in the composition.

38. A method according to claim 34 wherein the catalyst comprises about 4% to about 50% by weight of the metal phosphate as measured by amount of the metal's corresponding oxide present in the composition.

39. A method according to claim 34 wherein zeolite (a) is selected from ZSM-5, mordenite, ferrierite and any other zeolite having a silica to alumina molar ratio of twelve or greater.

40. A method according to claim 34 wherein the zeolite is ZSM-5.

41. A method according to claim 34 wherein the metal of (c) is selected from the group consisting of iron, lanthanide series and the cracked hydrocarbons produced by the method have enhanced propylene yields as measured by C₃/C₄ ratio compared to a catalyst composition that does not comprise the metal phosphate binder.

42. A method according to claim 34 wherein the metal of (c) is selected from the group consisting of Group IIA metals and the cracked hydrocarbons produced by the method have enhanced butylene yields as measured by C₃/C₄ ratio compared to a catalyst composition that does not comprise the metal phosphate binder.

43. A method according to claim 34 wherein the method of catalytic cracking is fluidized and the catalyst composition has a mean particle size in the range of 40 to about 150 microns.

44. A method according claim 34 wherein the method is a fixed bed catalytic cracking process and the catalyst composition is in the form of an extrudate.

45. A method according claim 34 wherein the method is a moving bed catalytic cracking process and the catalyst composition is in the form of an extrudate.

46. A method of making a catalyst composition, the method comprising
(a) combining a source of metal, other than aluminum, with zeolite
(b) adding phosphoric acid to (a)
(c) processing (b) under conditions sufficient to produce a bound composition comprising zeolite, and a phosphate of the metal from (a) wherein the metal phosphate is present in an amount sufficient to at least function as a binder for the zeolite.

47. A method according to claim 46 wherein the metal of (a) is selected from the group consisting of Group IIA metals, lanthanide series metals, scandium, yttrium, lanthanum, and transition metals.

48. A method according to claim 46 wherein the catalyst composition comprises at least 5% by weight of the phosphate of the metal from (a) as measured by amount of the metal's corresponding oxide present in the composition.

49. A method according to claim 46 where in the source of metal is in the form of a metal salt.

50. A composition comprising (a) a non-zeolitic molecular sieve, and (b) metal phosphate present in an amount sufficient for the metal phosphate to at least function as a binder for the non-zeolitic sieve and the metal is other than aluminum.

51. A composition according to claim 50 wherein the metal of (b) is selected from the group consisting of Group IIA metals, lanthanide series metals, scandium, yttrium, lanthanum, and transition metals.

52. A composition according to claim 50 wherein the nonzeolitic molecular sieve (a) is selected from the group consisting of SAPO, AlPO, and MCM-41.
